(19) United States
(12) Patent Application Publication
(10) Pub. No.: US 2025/0134781 A1
(43) Pub. Date: May 1, 2025

(54) HIGH STRENGTH LIFTING AND ANTIAGING FACIAL MASK WITH SKIN ACTIVES

(71) Applicant: L'OREAL, Paris (FR)

(72) Inventors: Yan WANG, Chatham, NJ (US); Catherine CHIOU, Saddle Brook, NJ (US); Yixin YANG, Union, NJ (US)

(73) Assignee: L'OREAL, Paris (FR)

(21) Appl. No.: 18/499,198

(22) Filed: Oct. 31, 2023

(51) Int. Cl. A61K 8/02; A61Q 19/08

(52) U.S. Cl. CPC A61K 8/0212; A61Q 19/08; A61K 2800/48; A61K 2800/882; A61K 2800/95

(57) ABSTRACT

A two-part composition for application to skin to forms a rinse-off high strength lifting and antiaging gel mask. The cosmetic composition is sensitive-skin friendly, and provides hydration benefits and soft focus/blurring effects, conferring transformative professional level results including skin smoothing, firming, texture improvement, and radiance boosting, which results are immediate and lasting beyond removal of the cosmetic composition from skin. The cosmetic composition that includes: Combinable parts including at least one silicate and water; and at least one cross-linked polyacrylic acid thickener; at least one clay; at least one glycosaminoglycan having a molecular weight in a range from about 1 million Daltons to about 1.4 million Daltons.

### FIELD OF THE INVENTION

The invention relates to a two-part composition for application to skin to form a rinse-off high strength lifting and antiaging gel mask.

### BACKGROUND OF THE INVENTION

Skin produces less collagen and elastin as it ages. For example, after the age of twenty, a person (human) produces about one percent less collagen in the skin each year. As a result, the skin becomes thinner and more fragile. Inevitably, dark spots, redness, wrinkles, crow's feet, age-spots, eye bags, and the like, may begin to form.

Consumers often wish to improve the appearance of such age-related skin imperfections, and seek instantaneous and lasting results. Many consumer products and procedures are devoted to hiding and reducing skin defects and imperfections. Some employ medical procedures while others include makeups, foundations, and masks, and topical products for providing hydration, such as lotions and creams. Some of these products contain active ingredients, for example, niacinamide, that help repair and rejuvenate skin over time, as well as peptides and plant extracts. Unfortunately, these products and procedures have drawbacks. For example, after removal of makeup type solutions, the skin looks the same as before the makeup was applied. In addition, makeup products are often visible, offer minimal texture benefits, and have no long-term lasting effect on the skin, thus making them less attractive as a solution for aging skin. Likewise, hydrating products do not address wrinkles and lines at least in the short term. And, of course, many consumers either cannot afford, or do not prefer to undergo such drastic cosmetic clinical procedures.

Attempts have been made to develop new categories of products to improve the appearance of skin without the drawbacks of existing products and procedures. One such family of products can be generally classified as “adhesive, contractile film formers.” Film formers are chemical compositions that when applied to skin, leave a pliable, cohesive, and continuous covering. A select group of film formers are also adhesive to the skin and contractile. Unfortunately, these film former type products, while providing dramatic, instant results, quickly lose their skin tightening effect, lose their elasticity, and can whiten, crack, and peel. In addition, such products may include harsh ingredients such as peptides and plant extracts that can be irritating, especially when combined with firm tightening agents, and can be difficult to apply to skin.

The instant disclosure provides an improved skin firming product that that is easy to apply, can be removed with water, does not cause skin irritation, and once removed provides immediate near term firming and longer term skin benefits.

### BRIEF SUMMARY OF THE INVENTION

The following summary is provided to introduce a selection of concepts in a simplified form that are further described below in the detailed description of the invention. This summary is not intended to identify key features of the claimed subject matter, nor is it intended to be used as an aid in determining the scope of the claimed subject matter.

In accordance with various embodiments, provided is a two-part composition for application to skin to form a rinse-off high strength lifting and antiaging gel mask. The composition provides the user with a fresh mixing experience prior to application, and upon use and removal by rinsing, provides immediate skin firming benefits and long-term skin smoothening results. The cosmetic composition is sensitive-skin friendly, and provides hydration benefits and soft focus/blurring effects, conferring transformative professional level results including skin smoothing, firming, texture improvement, and radiance boosting, which results are immediate and lasting beyond removal of the cosmetic composition from skin.

In some embodiments, the disclosure provides a cosmetic composition that includes: Combinable parts including a lifting serum comprising at least one silicate and water, and a gelling solution part comprising at least one cross-linked polyacrylic acid thickener, at least one clay, at least one glycosaminoglycan comprising a hyaluronic acid selected from sodium hyaluronate, hydrolyzed sodium hyaluronate, a hyaluronic acid derivative, or a combination thereof, the at least one glycosaminoglycan having a molecular weight in a range from about 1 million Daltons to about 1.4 million Daltons, wherein, upon mixing of the combinable lifting serum and gelling parts and application to a substrate, the composition forms a semi-solid gel film.

In some embodiments, the cosmetic composition is free or essentially free of ingredients that would appreciably alter the pH within the ranges as described herein, including acids and reactive ingredients that would produce acidic byproducts, as well as plasticizers such as glycerin (also identified herein as a hydrating agent or humectant). In some embodiments, the composition is free or essentially free of niacinamide. In some embodiments, the composition is free or essentially free of peptides. In some embodiments, the composition is free or essentially free of plant extracts, for example, but not limited to, Camellia Sinensis Leaf Extract, Nelumbo Nucifera (Lotus) Seed Extract, Sophora Angustifolia Root Extract, Rhus Semialata Extract, Ginkgo Biloba Leaf Extract, Lonicera Japonica (Honeysuckle) Flower Extract, Camellia Sinensis Leaf Extract, Portulaca Oleracea Extract, and combinations thereof.

In some embodiments, the at least one silicate is selected from the group consisting of sodium silicate, magnesium silicate, calcium silicate, aluminum silicate, and combinations thereof. In some embodiments, the silicate comprises sodium silicate.

In some embodiments, the at least one thickener comprises a carbomer.

In some embodiments, the at least one thickener comprises a carbomer and the at least one silicate comprises sodium silicate.

In some embodiments, the at least one glycosaminoglycan comprising a hyaluronic acid selected from sodium hyaluronate, hydrolyzed sodium hyaluronate, a hyaluronic acid derivative, or a combination thereof comprises sodium hyaluronate.

In some embodiments, the at least one clay is selected from the group consisting of hectorite, montmorillonite, bentonite, attapulgite, sepiolite, talc, mica, and combinations thereof.

In some embodiments, the pH is between about 9 and 11. In some embodiments the pH is between 10.5 and 11. In some embodiments, the pH is about 10.8.

In some embodiments, a ratio of cross-linked polyacrylic acid thickener to silicate is a range from about 1:1 to 1:8. In some embodiments, a ratio of polyacrylic acid thickener (for example, carbomer) to silicate is about 1:2.5.

In some embodiments, the cosmetic composition includes one or more one or more additives selected from salts, chelating agents, antimicrobial agents, neutralizing/pH-adjusting agents, surfactants, oils, waxes, butters, vitamins, fragrances, pearlescent agents, odor absorbers, coloring materials, essential oils, and emulsifiers, fruit extracts, skin actives, and combinations thereof.

In some embodiments, the at least one silicate is present in a range from about 1% to about 15%, the at least one thickener is present in a range from about 1% to about 2%, the at least one glycosaminoglycan comprising a hyaluronic acid is present is present in a range from about 0.01% to about 0.1%, the at least one clay is present in a range from about 0.02% to about 0.1%, and water is present in a range from about 89% to about 98%, all amounts by weight, based on the weight of the cosmetic composition.

In some embodiments, the disclosure provides a cosmetic composition, comprising: combinable parts including a lifting serum part that includes at least one silicate and water; and a gelling solution part that includes at least one cross-linked polyacrylic acid thickener, at least one clay, at least one glycosaminoglycan comprising a hyaluronic acid selected from sodium hyaluronate, hydrolyzed sodium hyaluronate, a hyaluronic acid derivative, or a combination thereof, the at least one glycosaminoglycan having a molecular weight in a range from about 1 million Dalton to about 1.4 million Dalton, wherein, upon mixing of the combinable parts and application to a substrate, the composition forms a semi-solid gel film, and wherein the at least one silicate comprises a sodium silicate present at about 3.3%, the at least one thickener comprises a carbomer, present at about 1.3%, the at least one glycosaminoglycan comprises sodium hyaluronate present at about 0.07%, the at least one clay comprises hectorite present at about 0.5% by weight, water is present at about 94% by weight, and the cosmetic composition includes butylene glycol is present at about 0.3%, all amounts by weight based on the weight of the composition.

In some such embodiments, the pH is about 10.8.

In some embodiments, the disclosure provides an article of manufacture comprising a composition according to any of the foregoing described compositions, wherein the article of manufacture includes a first container retaining the lifting serum part (i) of the composition and a second container retaining the gelling solution (ii) of the composition.

In some embodiments, the article of manufacture is characterized by one of the article of manufacture characterized by one of: (a) the first and second containers are provided for dispensing multiple doses of the composition, wherein one of the first and second containers is sized to receive the contained contents of both the first and second containers, such that the contained parts may be blended and used over time until the composition is exhausted; (b) the first and second containers are formed as discrete chambers of a single package have a frangible seal between them, such that the seal may be broken to permit mixing of the contents of the first and second containers, to thereby provide a single or plurality of doses; (c) the first and second containers are formed as discrete pouches of a single package; or (d) the first and second containers are formed as discrete pouches, jars, packets, compacts, bottles, tubes, or ampoules.

In some embodiments, the article of manufacture includes an applicator. In some embodiments, the applicator is a natural brush, a silicone brush, or a spatula.

In some embodiments, the disclosure provides a method of forming a gel mask on a substrate, comprising: providing a composition according to claim 1, mixing the component parts of the composition for 10-30 seconds; applying the mixture to the substrate for a period of time from about 10 to about 25 minutes; rinsing the substrate with warm water.

Other features and advantages of the present invention will be apparent from the following more detailed description, by way of example, the principles of the invention.

This disclosure describes particular embodiments in accordance with the general inventive concepts and is not intended to limit the scope of the invention in any way. Indeed, the invention as described in the specification is broader than and unlimited by the particular embodiments set forth herein, and the terms used herein have their full ordinary meaning.

### DETAILED DESCRIPTION OF THE INVENTION

In accordance with various embodiments, provided is a two-part cosmetic composition for application to skin to form a rinse-off gel mask. The cosmetic composition is sensitive-skin friendly, and provides hydration benefits and soft focus/blurring effects, conferring transformative professional level results including skin smoothing, firming, texture improvement, and radiance boosting, which results are immediate and lasting beyond removal of the cosmetic composition from skin.

As provided in the article of manufacture, the cosmetic composition provides a professional type of facial that is easy to use and apply and after gentle rinsing provides sustained benefits that closely match a competitive benchmark with a different compositional platform, and that edges out the competitive benchmark when comparing skin texture, radiance, and refinement.

In some embodiments, the disclosure the cosmetic composition includes: combinable parts including a first lifting serum part that includes at least one silicate and water; and a second gelling solution part that includes at least one cross-linked polyacrylic acid thickener; at least one clay; at least one glycosaminoglycan comprising a hyaluronic acid selected from sodium hyaluronate, hydrolyzed sodium hyaluronate, a hyaluronic acid derivative, or a combination thereof, the at least one glycosaminoglycan having a molecular weight in a range from about 1 million Daltons to about 1.4 million Daltons. The composition enables inclusion of skin actives at levels that confer skin benefits including soft focus blurring of skin with smoothness and sustained hydration after rinsing.

Upon mixing of the combinable parts and application to a substrate, the silicate and polymer film formers in the composition form a semi-solid gel film that may be applied with ease after mixing and retained on the skin without dripping until gently removed with water. Without being bound by theory, the mixing of the lifting serum part with the gelling solution triggers thickening of the at least one cross-linked polyacrylic acid thickener (exemplified herein as carbomer) which initiates sol gel transition of sodium silicate. Within a pH range from about 9-11, and in particular embodiments at a pH of about 10.8, the two parts form a silica gel that confers transformative texture that instantly lifts skin and upon rinsing, unexpectedly confers lasting effects. In various embodiments, the cross-linked polyacrylic acid thickener and silicate ingredients are provided in the composition in an advantageous ratio that allows formation of a silica gel that is spreadable on skin after mixing and firm enough to be retained on skin until rinsing, wherein the ratio of cross-linked polyacrylic acid thickener to silicate is a range from about 1:1 to 1:8, in some embodiments about 1:2 to about 1:3, or about 1:2.5.

In some embodiments, the cosmetic composition is free or essentially free of ingredients that would appreciably alter the pH, as described herein, including acids and reactive ingredients that would produce acidic byproducts, plasticizers such as glycerin (also identified herein as a hydrating agent or humectant). In some embodiments, the composition is free or essentially free of niacinamide. In some embodiments, the composition is free or essentially free of peptides. In some embodiments, the composition is free or essentially free of plant extracts, for example, but not limited to, Camellia Sinensis Leaf Extract, Nelumbo Nucifera (Lotus) Seed Extract, Sophora Angustifolia Root Extract, Rhus Semialata Extract, Ginkgo Biloba Leaf Extract, Lonicera Japonica (Honeysuckle) Flower Extract, Camellia Sinensis Leaf Extract, Portulaca Oleracea Extract.

In some embodiments, the inventive composition is used on one or more of the parts of or the entire body, the face, the hair and/or scalp, and discrete body parts. The cosmetic composition may be used on “normal” skin or that is other than normal, and for example, may be dry, or oily.

### Lifting Serum Part

### Silicate

In accordance with the disclosure, the cosmetic composition includes at least one silicate.

In various embodiments, the at least one silicate is selected from the group consisting of sodium silicate, magnesium silicate, calcium silicate, aluminum silicate, and combinations thereof. In some particular embodiments, the silicate is sodium silicate. Examples of silicates that may be obtained commercially include sodium silicate hydrous powders that are soluble in water, including those selected from BRITESIL C 205, BRITESIL C207, from PQ Corporation, and combinations thereof. In some embodiments, the at least one silicate includes BRITESIL C 205.

In some embodiments, the at least one silicate comprises a sodium silicate. In some embodiments, the sodium silicate is present in a range from about 1% to about 15%, or at about 3.3%, by weight, based on the weight of the cosmetic composition. In some embodiments, the sodium silicate is represented by the formula Na2O.(SiO2)x, where x is the ratio of silicon dioxide (SiO2) to sodium oxide (Na₂O) by weight, wherein the silicon dioxide (SiO₂) is present with sodium oxide (Na₂O) at a weight ratio (“x”) in a range from about 2.0 to about 3.4, or at about less than 2.4.

In various embodiments, the total amount of silicate present in the cosmetic composition is in an amount from about 1% to about 20%, or from about 1% to about 15%, or from about 1% to about 9%, or from about 2% to about 4%, or any suitable combination, sub-combination, range, or sub-range thereof by weight, based on the weight of the cosmetic composition. One of ordinary skill in the art, however, will appreciate that other ranges are within the scope of the invention.

Thus, the at least one silicate is present, by weight, based on the total weight of the composition, from about 1.0, 2, 3, 4, 5, 6, 7, 8, 9, 10, 11, 12, 13, 14, 15, 16, 17, 18, 19 to about 20 percent, including increments and ranges therein and there between.

### Solvent/Water

In accordance with the various embodiments, water is present in the cosmetic compositions in a range from about 80% to about 98%, or from about 85% to about 95%, or from about 89% to about 95%, or any suitable combination, sub-combination, range, or sub-range thereof by weight, based on the weight of the cosmetic composition. In some embodiments, water is present in the cosmetic composition in an amount from at least about 94% by weight, based on the weight of the cosmetic composition. One of ordinary skill in the art, however, will appreciate that other ranges are within the scope of the invention. Thus, water is present, by weight, based on the total weight of the cosmetic composition, from about 80, 81, 82, 83, 84, 85, 86, 87, 88, 89, 90, 91, 92, 93, 94, 95, 96, 97, to about 98 weight percent, including increments and ranges therein and there between.

In some embodiments, water is present in a range from about 89% to about 98%, or at about 94% by weight, based on the weight of the cosmetic composition.

The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-saunier, water from Eaux Bonnes, water from Rochefort, water from Saint Christau, water from Les Fumades, water from Tercis-les-Bains or water from Avene. The water phase may also comprise reconstituted thermal water, which is to say a water comprising trace elements such as zinc, copper, magnesium, etc., reconstituting the characteristics of a thermal water.

In some embodiments, the cosmetic composition can include one or more additional water soluble solvents. Non-limiting examples of water-soluble solvents include, for example, glycerin, C1-4 alcohols, organic solvents, fatty alcohols, fatty ethers, fatty esters, polyols, glycols, vegetable oils, mineral oils, liposomes, laminar lipid materials, or any a mixture thereof. As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol.

In some embodiments, the cosmetic composition may be free or essentially free of one or more plasticizers, for example, plasticizers selected from the group consisting of phthalate esters, adipates, citrates, alkane-dicarboxylic, glycols and phosphates. Thus, in some examples, the composition may be free of glycerin, propylene glycol, polyethylene glycol, glycerol, sorbitol, dipropylene glycol, propane diol, triethyl citrate, isohexadecane, and combinations thereof.

When present, the total amount of one or more water-soluble solvents may be about 0.1% to about 20%, or from about 0.1% to about 15%, or from about 0.1% to about 10%, or from about 0.1% to about 0.5%, %, or any suitable combination, sub-combination, range, or sub-range thereof by weight, based on the total weight of the cosmetic composition.

In some embodiments, the cosmetic composition includes butylene glycol. In some embodiments, butylene glycol is present at about 0.3% by weight based on the weight of the composition.

### pH of the Cosmetic Composition

The pH of the cosmetic composition is between 9 and 11, or between 9.5 and 11, or between 10.2 and 11, or between 10.5 and 11 and in some embodiments, is about 10.8. The pH can be adjusted to the desired value by addition of a base (organic or inorganic) to the cosmetic composition, for example ammonia or a primary, secondary or tertiary (poly) amine, such as monoethanolamine, diethanolamine, triethanolamine, isopropanolamine or 1,3-propanediamine, or alternatively by addition of an inorganic or organic acid, advantageously a carboxylic acid, such as, for example, citric acid.

As disclosed herein, the pH can also be selected by varying the proportions of the gelling part comprising the at least one cross-linked polyacrylic acid thickener (in some embodiments carbomer) with the lifting serum part comprising the silicate (in some embodiments sodium silicate).

Accordingly, the pH may be in a range from about 9 to about 11, including increments of 0.01 therein and therebetween, and in some examples, the pH may be in a range from about 10.50, 10.51, 10.52, 10.53, 10.54, 10.55, etc to about 10.81, 10.82, 10.83, 10.84, 10.85 etc to about 11.00.

Maintaining the pH in the described range is influenced by the relative proportions of thickener comprising polymer and silicate. In view of the relatively high pH of silicate as compared to that of the thickener, an imbalance in these ingredients can result in the composition becoming cloudy and gummy or unusable. Accordingly, the ratio of cross-linked polyacrylic acid thickener (e.g., carbomer) to silicate (e.g., sodium silicate) is the range from about 1:1 to 1:8, including increments and ranges therein and there between. In some embodiments, the ratio is the range from about 1:1 to 1:4, or from about 1:1.5 to about 1.3.5, or about 1:3, and in some examples about 1:2.6. Variously, the ratio may be 1:1, 1:2, 1:3, 1:4, 1:5, 1:5, 1:7, to 1:8, including increments and ranges therein and there between, based on the total weight of the cosmetic composition

### Gelling Solution Part

### Thickener (Carbomer)

In accordance with the disclosure, the cosmetic composition includes at least one thickener.

In some particular embodiments, the thickener is a cross-linked polyacrylic acid, for example, a carbomer. Examples of cross-linked polyacrylic acid thickeners may be obtained commercially from Lubrizol™ as one of Carbopol Ultrez™—Carbopol Ultrez 10, 21, 30, as a cross-linked polyacrylic acid polymerized and provided in a cosolvent system and offers good electrolyte tolerance and thickening performance in a broad pH range, for example wherein the pH is between 9 and 11, and in some embodiments, about 10.8.

In some embodiments, the at least one thickener comprises a carbomer. In some embodiments, the carbomer is present in a range from about 1% to about 2%, or at about 1.3%, by weight, based on the weight of the cosmetic composition.

In various embodiments, the total amount of thickener present in the cosmetic composition is in an amount from about 0.5% to about 3%, or from about 1% to about 2%, or any suitable combination, sub-combination, range, or sub-range thereof by weight, based on the weight of the cosmetic composition. In some embodiments, the total amount of thickener comprising a cross-linked polyacrylic acid is present up to and not greater than about 2% by weight, based on the weight of the cosmetic composition. One of ordinary skill in the art, however, will appreciate that other ranges are within the scope of the invention.

Thus, one or more thickener, is present by weight, based on the total weight of the cosmetic composition, from about 0.5, 0.6, 0.7, 0.8, 0.9, 1.0, 1.1, 1.2, 1.3, 1.4, 1.5, 1.6, 1.7, 1.8, 1.9, to about 2.0 percent, including increments and ranges therein and there between.

### Glycosaminoglycan (Hyaluronic Acid)

In accordance with the disclosure, the cosmetic composition includes at least one glycosaminoglycan comprising a hyaluronic acid selected from sodium hyaluronate, hydrolyzed sodium hyaluronate, a hyaluronic acid derivative, or a combination thereof.

In some embodiments, the at least one glycosaminoglycan comprising a hyaluronic acid selected from sodium hyaluronate, hydrolyzed sodium hyaluronate, a hyaluronic acid derivative, or a combination thereof comprises sodium hyaluronate. In some embodiments, the sodium hyaluronate is present is present in a range from about 0.01% to about 0.1%, or at about 0.07% by weight, based on the weight of the cosmetic composition.

In some embodiments, the at least one glycosaminoglycan selected from sodium hyaluronate, hydrolyzed sodium hyaluronate, a hyaluronic acid derivative, or a combination thereof has a molecular weight in a range from about 1 million Daltons to about 1.4 million Daltons. In some particular embodiments, the at least one glycosaminoglycan comprising a hyaluronic acid is sodium hyaluronate having a molecular weight of about 1 million Daltons to about 1.4 million Daltons.

Examples of sodium hyaluronate that may be obtained commercially include Cristalhyal LO™ from Givaudan Active Beauty.

The at least one glycosaminoglycan comprising a hyaluronic acid selected from sodium hyaluronate, hydrolyzed sodium hyaluronate, a hyaluronic acid derivative, or a combination thereof in the cosmetic composition is present in an amount from about 0.01% to about 0.3%, or from about 0.03% to about 0.25%, or from about 0.05% to about 0.2%, or from about 0.06% to about 0.1%, or from about 0.065% to about 0.08%, or at least about 0.02% by weight, or at least about 0.05% by weight, or at least about 0.075% by weight, or at least about 0.1% by weight, or at least about 0.2% by weight, or any suitable combination, sub-combination, range, or sub-range thereof by weight, based on the weight of the cosmetic composition. One of ordinary skill in the art, however, will appreciate that other ranges are within the scope of the invention.

Thus, the at least one glycosaminoglycan comprising a hyaluronic acid selected from sodium hyaluronate, hydrolyzed sodium hyaluronate, a hyaluronic acid derivative, or a combination thereof, is present in an amount by weight, based on the total weight of the cosmetic composition, from about 0.01, 0.02, 0.03, 0.04, 0.05, 0.06, 0.07, 0.08, 0.09, 0.10, 0.20, to about 0.30 weight percent, including increments and ranges therein and there between.

### Clay

In accordance with the disclosure, the cosmetic composition includes at least one clay. A single type of clay may be used, or two or more different types of clays may be used in combination.

In some embodiments, the at least one clay may be an organophilic clays, more particularly modified clays, such as hectorite, montmorillonite, bentonite, attapulgite and sepiolite, and mixtures thereof. These clays are modified with a chemical compound chosen from quaternary amines, tertiary amines, amine acetates, imidazolines, amine soaps, fatty sulfates, alkylarylsulfonates and amine oxides, and mixtures thereof.

In some embodiments, the at least one clay is selected from the group consisting of talc, mica and hectorite.

In some embodiments, the at least one clay comprises hectorite. In some embodiment, the at least one clay consists of hectorite present in a range from about 0.02% to about 0.1%, or at about 0.5% by weight, based on the weight of the cosmetic composition.

Examples of hectorite that may be obtained commercially include BENTONE HYDROCLAY 2000 LO from Elementis. Other examples include BENTONE HYDROCLAY 2000, BENTONE HYDROCLAY 1100, BENTONE HYDROCLAY 2001, BENTONE HYDROCLAY 2100, BENTONE HYDROCLAY 900, or a combination thereof. In a particular embodiment, BENTONE HYDROCLAY 2000 may be selected.

In various embodiments, the total amount of at least one clay present in the cosmetic composition is in an amount from about 0.01% to about 0.2%, or from about 0.02% to about 0.1%, or from about 0.03% to about 0.08%, or from about 0.04% to about 0.05%, or at least about 0.02% by weight, or at least about 0.05% by weight, or any suitable combination, sub-combination, range, or sub-range thereof by weight, based on the weight of the cosmetic composition. One of ordinary skill in the art, however, will appreciate that other ranges are within the scope of the invention.

Thus, each of the at least one clay is present in an amount by weight, based on the total weight of the cosmetic composition, from about 0.01, 0.02, 0.03, 0.04, 0.05, 0.06, 0.07, 0.08, 0.09, 0.10, to about 0.2 weight percent, including increments and ranges therein and there between.

The term “clay” refers to a naturally occurring material composed primarily of fine-grained minerals, which is generally plastic at an appropriate water content and will harden when dried or fired. Although clay usually contains phyllosilicates, it may contain other materials that impart plasticity and harden when dried or fired. Associated phases in clay may include materials that do not impart plasticity and organic matter. A common definition is that in the Penguin Dictionary of Science, namely “finely divided rock materials whose component minerals are various silicates, mainly of magnesium or aluminium.” Clay comprises Kaolinite (typically defined as [Si₄]Al₄O₁₀(OH)₈.nH₂O (n=0 or 4)), Illite (typically defined as Mₓ[Si₆₋₈Al₁₋₂]Al₃Fe_{.025}Mg_{.75}O₂₀ (OH)₄), Vermiculite (typically defined as Mₓ[Si₇Al]AlFe_{.05}Mgo._{.5}O₂₀ (OH)₄), Smectite (typically defined as MX[Si₈]Al_{3.}²Fe₀₋₂Mg₀₋₆O₂₀ (OH)₄, Chlorite (typically defined as (Al(OH)_{2.55})4[Si₆₋₈AlO₁₋₂}Al₃₋₄Mg₀₋₆)₂₀(OH)₄), and Phyllosilicate minerals or talc (typically defined as Mg₃Si₄O₁₀(OH)₂).

Another definition, frequently used by chemists is “a naturally occurring sediment or sedimentary rock composed of one or more minerals and accessory compounds, the whole usually being rich in hydrated aluminum silicate, iron or magnesium, hydrated alumina, or iron oxide, predominating in particles of colloidal or near-colloidal size, and commonly developing plasticity when sufficiently pulverized and wetted” (see Kirk-Othmer, Encyclopaedia of Chemical Technology, Volume 5, page 544, 2nd edition, John Wiley and Sons, Inc., New York, N.Y. 1964). Example of clays are given in the book “Clay mineralogy, S. Caillere, S. Henin, M. Rautureau, 2nd edition 1982, Masson.” Clays may be of natural or synthetic origin.

Hydrophilic clay includes smectites such as saponites, hectorites, montmorillonites, bentonites, beidellite. Hydrophilic clay includes synthetic hectorites (also called laponites) such as the products sold by the company under the name Laporte Laponite XLG, Laponite RD, Laponite RDS (these products are sodium silicates and magnesium silicates in particular sodium, lithium and magnesium) bentonites such as the product sold under the name Bentone (Registered TM) HC Rheox, magnesium silicates and aluminum products such as hydrated products sold by Vanderbilt Company as ultra Veegum (Registered TM), Veegum (Registered TM) HS, Veegum (Registered TM) DGT, or calcium silicates, particularly the synthetic form sold by the company under the name Micro-Cel (Registered TM) C. Fuller's earth consists chiefly of hydrated aluminum silicates that contain metal ions such as magnesium, sodium, and calcium within their structure. Montmorillonite is the principal clay mineral in fuller's earth, but it may contain other minerals such as kaolinite, attapulgite, and palygorskite among other components.

Lipophilic clay means clay swellable in a lipophilic medium, the clay swells and forms a colloidal dispersion. Lipophilic clays include modified clays such as the modified magnesium silicate (Bentone gel VS38 from Rheox) hectorites modified with an ammonium chloride fatty acid CIO to C22, such as hectorite modified with ammonium chloride distearyldimethylammonium (CTFA name: Disteardimonium hectorite) sold under the name “Bentone 38 CE” by Rheox or Bentone (Registered TM) 38V by ELEMENTIS.

The origin of such clay can be natural or synthetic mineral clay such as hectorite, bentonite, and quaternized derivatives thereof, for example which are obtained by reacting the minerals with a quaternary ammonium compound, such as stearalkonium bentonite, hectorites, quaternized hectorites such as Quatemium-18 hectorite, carbonates such as propylene carbonate, bentones, and the like.

The non-limiting of examples of clay which can be used in the present invention are Fuller's earth, Pinatubo volcanic ash mud from Philippines, Aleppo clay from Syria, Pulau tiga volcano mud from Malasiya, Nha Trang mud from Vietnam, White Kaolinite from Korea, Yellow Loess from Korea, Jeju volcanic clay from Korea, Guanziling mud form Taiwan, Wudalianchi volcanic mud from China, Black mud of Yuncheng salt lake from China, mineral mud from Tantou village in China, China clay (Kaolin), Maifan stone from China, Beppu onsen Fango from Japan, Kucha from Japan, Tanakura clay from Japan, Cambrian blue clay from Russia, Blue Lagoon mud from Iceland, Saki lake mud from Ukraine, Karlovy Vary moor mud from Czech Republic, Heviz Georgikon moor mud from Hungry, Alpine moor mud from Austria, Bad Wilsnack mud from Germany, Bavarian mineral slat mountain mud from Germany, Freiburg volcanic ash from Germany, Santorini mud from Greece, Mar Menor mud from Spian, Ischian volcanic mud from Italy, Euganean thermal mud from Italy, Yellow clay-Illite from France, French Green Clay—Montmorrillonite, Calistoga mud from USA, Sacred clay and ormalite from USA, Redmond clay from USA, Arctic mineral mud from Canada, Tulum Mayan clay from Mexico, Glacial clay from Canada, Amazonian white clay from Brazil, El Chillante volcanic thermal mud from Argentina, African healing clay, Australian olive green clay.

### Other Ingredients

In accordance with the disclosure, the cosmetic composition may include other ingredients. In some embodiments, some optional ingredients are expressly excluded from the composition.

### Optional Additives

The cosmetic compositions can also comprise one or more additives selected from salts, chelating agents, antimicrobial agents, neutralizing/pH-adjusting agents, surfactants, oils, waxes, butters, vitamins, fragrances, pearlescent agents, odor absorbers, coloring materials, essential oils, and emulsifiers, fruit extracts, skin actives, and combinations thereof.

In some embodiments, additives may include one or a combination of chelating agents, selected from, for example, sodium phytate, ethylenediaminetetraacetic acid (EDTA), tetrasodium etidronate, tetrasodium pyrophosphate, pentasodium ethylenediamine tetramethylene phosphonate, sodium staminate, and combinations thereof. In some embodiments, additives may include one or a combination of humectants/hydrating agents, selected from, for example, glycerin, glycerol, butylene glycol, propylene glycol, isoprene glycol, dipropylene glycol, hexylene glycol and polyethylene glycols, monoethylene glycol, diethylene glycol, triethylene glycol, diethylene glycol, hexylene glycol, glycol ethers such as monopropylene, dipropylene and tripropylene glycol alkyl(C1-C4) ethers, squalane, triacetin, sugars, such as glucose, xylitol, maltitol, sorbitol, sucrose pentaerythritol, inositol, pyrrolidone carboxylic acid, lactic acid, lithium chloride, acetamide MEA, sodium lactate, urea, dicyanamide, aloe vera, honey, and seaweed extract, and combinations thereof.

In some embodiments, additives may include one or a combination of antimicrobial agents and their salts, selected from, for example, phenoxyethanol, chlorphenesin, caprylyl glycol, caprylhydroxamic acid, benzoic acid, salicylic acid, benzyl alcohol, phenethyl alcohol, benzalkonium chloride, 4-hydroxyacetophenone, piroctone olamine, hexyl glycerin, ethylhexylglycerin, octylglycerin, benzylglycerin, 3-heptoyl-2,2-propandiol, and 1,2-hexandiol, and pentylene glycol. In some embodiments, additives may include surfactants selected from anionic surfactants, amphoteric/zwitterionic surfactants, and non-ionic surfactants.

In some embodiments, skin actives may include anti-aging compounds, ultraviolet (UV) active compounds, anti-acne compounds, anti-perspiration compounds, anti-pigmentation compounds, anti-inflammation compounds, antioxidants (e.g., EDTA, BHT, tocopherol), or a combination thereof. Suitable examples of active compounds include, but are not limited to, caffeine, adenosine, Scutellaria Baicalensis root extract, resveratrol, tocopherol, hydroxypropyl tetrahydropyrantriol, terephthalylidene dicamphor sulfonic acid, and combinations thereof.

In some particular embodiments, the cosmetic composition may include as additives phenoxyethanol and caffeine. In some embodiments, phenoxyethanol and caffeine are present at about 0.7% and 0.4%, respectively.

In accordance with the various embodiments, the amount of one or more additives, alone or in combination, may be present in the cosmetic composition according to the disclosure in a range from about 0.001% to about 20%, by weight, or from about 0.005% to about 0.01%, or from about 0.01% to about 0.1%, or from about 0.15% to about 5%, or from about 0.1% to about 0.5%, or from about 0.4% to about 4%, or from about 0.5% to about 2.5% by weight, or from about 1% to about 2%, or any suitable combination, sub-combination, range, or sub-range thereof by weight, based on the total weight of the cosmetic composition.

Thus, any one or a combination of additives may be present, by weight, based on the total weight of the cosmetic composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 weight percent, including increments and ranges therein and there between.

In some embodiments, the inventive composition may include one or more additional optional ingredients, for example, selected from but not limited to, silicones, including but not limited to silicone polymers, silicon elastomers, and dimethicone and other silicone oils, sulfates including but not limited to sulfate based.

In some embodiments, the inventive composition may exclude, be free from or devoid of one or more ingredients, including ingredients as described herein and other ingredients, for example, selected from but not limited to: plasticizers, for example, glycerin, propylene glycol, polyethylene glycol, glycerol, sorbitol, dipropylene glycol, propane diol, triethyl citrate, isohexadecane, and combinations thereof; peptides, including but not limited to hexapeptides, tripeptides, tetrapeptides and polylysine, more particularly, acetyl hexapeptides (e.g., acetyl hexapeptide -3, -37, -38, and -51), acetyl tetrapeptides (e.g., acetyl tetrapeptide-2, -3, -5, -7, -8, -9, -11, and -15), RH-oligopeptide-1 (an epidermal growth factor), acetyl hexapeptide-8, copper tripeptide-8, tetrapeptide-21, acetyl dipeptide-1 cetyl ester, palmitoyl tripeptide-1, acetyl tetrapeptide-9, palmitoyl tetrapeptide-7, palmitoyl tripeptide-8, dipeptide diaminobutyroyl benzylamide diacetate, or a combination thereof; silicones, including but not limited to silicone polymers, silicon elastomers, and dimethicone and other silicone oils; sulfates including but not limited to sulfate based surfactants; alcohols; polyols; UV filters; oils; chelating agents; antimicrobial agents; neutralizing/pH-adjusting agents; vitamins; fragrances; pearlescent agents; odor absorbers; coloring materials; essential oils; fruit extracts; and combinations thereof.

In some embodiments, the cosmetic composition is free or essentially free of ingredients that would appreciably alter the pH, as described herein, including acids and reactive ingredients that would produce acidic byproducts, plasticizers such as glycerin (also identified herein as a hydrating agent or humectant). In some embodiments, the composition is free or essentially free of niacinamide.

In some embodiments, the composition is free or essentially free of plant extracts, for example, but not limited to, Camellia Sinensis Leaf Extract, Nelumbo Nucifera (Lotus) Seed Extract, Sophora Angustifolia Root Extract, Rhus Semialata Extract, Ginkgo Biloba Leaf Extract, Lonicera Japonica (Honeysuckle) Flower Extract, Camellia Sinensis Leaf Extract, Portulaca Oleracea Extract, and combinations thereof.

In some particular embodiments “free” means that the cosmetic composition is free, essentially free or devoid specifically of an excluded ingredient.

Those of skill in the art will appreciate that an excluded ingredient may be present in a composition via its presence in one or more of the formulation components but is otherwise not deliberately added to the cosmetic composition; thus, in some embodiments, a composition may be “essentially-free” wherein an excluded ingredient is present at a concentration that does not exceed 5% by weight, and in some instances is present not more than 3% by weight, and in some instances is present not more than 1% by weight, and in some instances is present not more than 0.5% by weight, and in some instances is present not more than 0.1% by weight, based on the weight of the cosmetic composition.

### Packaging

In various embodiments, the cosmetic composition is provided as an article of manufacture in which the ingredients are partitioned into at least two parts, such that gelling solution that includes the cross-linked polyacrylic acid thickener (e.g., carbomer), clay comprising hectorite, glycosaminoglycan, and any actives are retained separately from the lifting serum that includes silicate and water.

It will be appreciated that the cosmetic composition is packaged in an article of manufacture that separately contains each of the gelling solution part and the lifting serum part. In some embodiments, the article of manufacture includes each of the separately packaged parts having concentrations of the thickener and the silicate, respectively, that provide for a ratio of cross-linked polyacrylic acid thickener to silicate in a range from about 1:1 to 1:8, such that upon mixing thereof, the resulting composition has a predetermined pH in a range from about 9 to about 11, or between 10.5 and 11, or about 10.6 to about 10.8, and in some embodiments, about 10.83, wherein the pH is determined at least in part by the proportions of the thickener and silicate. Variation of the amounts of blended parts provide for a mask having varied thickness and spreadability, ranging from thinner and liquidous at a higher pH within the range from about 9 to 11 (greater amount of silicate within the range from 1:1 to 1:8) to thicker and firmer at a lower pH within the range from about 9 to 11 (greater amount of thickener within the range from 1:1 to 1:8).

In one embodiment, the packaging may be in the form of a multiuse product in which each of the two parts are separately provided wherein the container for at least one part is sized to receive the entirety of the other contained part, such that the parts may be blended by the consumer and used over time until the mixture is exhausted.

In another embodiment, the packaging may be in the form of a single use product wherein the two parts provided in separate containers or chambers, and the separately contained parts of a pack are blended by the consumer and used for a single or limited number of applications. Such embodiment may be a dual chamber packet having discretely isolated segments with an internal frangible thermoform seal therebetween that may be ruptured to mix the two parts in the packet prior to application. Alternatively, such embodiment may be a dual chamber pouch with adjacent segments, wherein each of the pouch segments are torn and the contents thereof are mixed in another container or on a substrate surface prior to application, or the two parts are mixed on the skin surface. Alternatively, such embodiment may be embodied as a single chamber pouch containing one part and a separate bottle, tube or ampoule containing the other part wherein the pouch segment is torn and the separate bottle, tube or ampoule is broken and the contents thereof are mixed in the pouch, another container or on a substrate surface prior to application, or the two parts are mixed on the skin surface. Alternatively, such embodiment may be embodied as a single chamber jar, packet or compact containing one part and a separate bottle, tube, applicator with a retaining compartment, or ampoule containing the other part wherein the jar, packet or compact is opened and the contents of the separate bottle, tube, applicator with a retaining compartment, or ampoule is added to and mixed in the single chamber jar, packet or compact, prior to application.

The article of manufacture may further include an applicator such as a pad, a brush or a spatula. Such articles may be made of natural materials and fibers, or silicone, rubber or plastic or metal. In some embodiments, the applicator is a natural bristle brush or a silicone brush.

In another embodiment, the packaging may be in the form of a multiuse product including a plurality of packs, according to one of the embodiments described above.

### Use of the Packaged Composition

In use, the article of manufacture comprising at least two partitioned containers is provided for use by a consumer, whereby the consumer opens the containers, mixes the component parts, applies the composition to skin, and maintains it in place for a time sufficient to allow gelling and tensing of the mask composition, for example, for an interval of about 30 minutes, or more generally in a range from about 10 minutes to about 45 minutes.

Application to the skin may be accomplished using fingers, an applicator pad, or a brush.

Articles of manufacture providing the composition may thus include partitioned containers for the first part and second parts comprising, respectively, the lifting serum and the gelling solution, and one or more applicators.

Accordingly, the component parts of the composition are mixed, for example for about, 10-30 seconds; the mixture is applied to the skin for a period of time from about ten to about 45 minutes; the skin is rinsed with warm water without use of any cleanser. Following removal of the mask, the skin may be further treated to a moisturizer and/or a makeup composition, UV protector, or other compositions may be applied.

Used according to the disclosure, the composition may be applied with ease to form a gel mask which confers immediate skin firming, and upon removal of the mask, the clay (e.g., hectorite) is retained to provide smoothing and soft focus blurring of lines and wrinkles, with the conferred effect of the mask providing enhanced skin hydration, and benefits of included actives, such as reduced redness with caffeine and hydrating and plumping with hyaluronic acid/sodium hyaluronate.

### EXAMPLES

### Example 1: Raw Materials

Below is a listing of raw materials included in the exemplified inventive composition.

TABLE 1
Raw Materials

| | | |
|---|---|---|
| | Commercial | Percent |
| Ingredient/INCI | Name | active if <100% |

| | |
|---|---|
| Sodium Silicate | BRITESIL C 205 (TM) |
| Carbomer | Carbopol Ultrez |
| | (TM) 10, 21, 30 |
| Hectorite | BENTONE HYDROCLAY |
| | 2000 LO (TM) |
| Sodium | Cristalhyal |
| Hyaluronate | LO (TM) |

### Example 2: Inventive Composition

A representative embodiment of the inventive composition is exemplified herein in Table 2. In the exemplified embodiments, the cosmetic composition is provided as a two part composition wherein the first part has a thick, honey-like consistency, and wherein the second part has a watery, serum-like consistency.

TABLE 2
Inventive Composition

| | % by weight of the | Part (as separately |
|---|---|---|
| INCI US | composition | packaged) |
| CARBOMER | 1.28 | 1 |
| HECTORITE | 0.05 | 1 |
| SODIUM HYALURONATE | 0.07475000 | 1 |
| PHENOXYETHANOL | 0.67 | 1 |
| CAFFEINE | 0.37 | 1 |
| BUTYLENE GLYCOL | 0.37 | 1 |
| SODIUM SILICATE | 3.32 | 2 |
| WATER | QS (~94) | 2 |

### Solutions of Gelling Solution and Lifting Serum

Below are representative examples of the two parts of the inventive composition, wherein in some examples, embodiments of the inventive composition may be prepared by mixing two parts, such as those exemplified below, in varying proportions to provide a composition having a pH in a range from about 9 to about 11, or between 10.5 and 11, or about 10.6 to about 10.8, and in some embodiments, about 10.83, wherein the Gelling Solution part and the Lifting Serum parts are blended to provide a ratio of cross-linked polyacrylic acid thickener to silicate is a range from about 1:1 to 1:8, or about 1:2.5.

TABLE 3
Gelling Solution Part and Lifting Serum Part

Inventive Example Gelling
and Lifting Parts

| | Gelling | Lifting |
|---|---|---|
| | Solution | Serum |
| Ingredient | Part | Part |
| BUTYLENE GLYCOL | ~0.5 | |
| HECTORITE | 0.07 | |
| PHENOXYETHANOL | 0.9 | |
| SODIUM SILICATE | | 13.15 |
| CAFFEINE | ~0.5 | |
| WATER | ~96 | ~87 |
| CARBOMER | 1.715 | |
| SODIUM HYALURONATE | 0.1 | |

### Example 3: Comparative Commercial Composition (Benchmark)

A comparative composition includes a similar format of a gel based mask that includes Water, Carbomer, Camellia Sinensis Leaf Extract, Nelumbo Nucifera (Lotus) Seed Extract, Yeast Beta-Glucan, Caffeine, 1,2-Hexanediol, Caprylyl Glycol, Butylene Glycol, Sophora Angustifolia Root Extract, Rhus Semialata Extract, Ginkgo Biloba Leaf Extract, Lonicera Japonica (Honeysuckle) Flower Extract, p-Anisic Acid, Acetyl Hexapeptide-8, Copper Tripeptide-1, Dipeptide-10, Dipeptide-15, Palmitoyl Tripeptide-1, Palmitoyl Tetrapeptide-7, Nicotinoyl Dipeptide-22, Nicotinoyl Dipeptide-23 Water, Sodium Silicate, Camellia Sinensis Leaf Extract, Portulaca Oleracea Extract, Polylysine. (References for this benchmark include Korean patent document KR20190001136A, and at URL hanacure.com/products/the-all-in-one-facial-set?variant=30803287436).

### Example 4: Consumer Study with Inventive and Comparative Compositions

A consumer study was conducted with each of the exemplified inventive and comparative compositions. Each of the inventive and comparative compositions were applied to different areas of the skin of each subject according to the method of use described above, and the subjects used the compositions daily for a period of four weeks. Test subjects reported that the product was easy to open and mix, and was easy to apply (See Table 4, below). Immediately after use the subjects reported that they could see and feel the product working on their skin with a notably enhanced benefit as compared to the benchmark (See Tables 4 and 5, below). After 2 and 4 weeks of use, test subjects reported an overall advantage of the inventive composition on texture, radiance and refinement of skin parameters (See Tables 4, 6 and 7, below) as compared with the comparative composition. Overall, the inventive composition outperformed the benchmark on mixing, ease of application, and immediate results. While long term results were comparable, after four weeks of use, test subjects reported comparatively better skin texture, radiance, and refinement vs benchmark. The results are summarized below. (*denotes test subject reported notable advantage)

TABLE 4
Overall Use Consumer Score (Score from 1-2)

| METRIC | INVENTIVE | BENCHMARK |
|---|---|---|
| Final Results After 4 weeks | 2 | 2 |
| Immediate Results after First Use | 2 | 1 |
| Mixing | 2 | 1 |
| Application | 2 | 2 |
| Drying | 1 | 1 |
| Removal | 1 | 1 |
| Sensorial Experience | 1 | 1 |

TABLE 5
Immediate Results Skin Benefits Score
(0-7, directional; not statistical)

| METRIC | INVENTIVE | BENCHMARK |
|---|---|---|
| SMOOTHER SKIN | 7.0* | 5.0 |
| MORE FIRM SKIN | 6.3 | 5.8 |
| HEALTHIER LOOKING SKIN | 6.5* | 1.8 |
| MORE RADIANT SKIN | 5.6* | 3 |
| IMPROVEMENT OVERALL TEXTURE | 4.7* | 1.8 |
| HYDRATED/MOISTURIZED | 4.9 | 3.2 |
| LOOK AND FEEL | | |
| COMFORTABLE/ | 3.0 | 3.6 |
| PLEASANT FEELING | | |
| MORE PLUMP/SOFT SKIN | 4.7 | 4.6 |
| LESS DULL SKIN APPEARANCE | 4.6* | 0 |
| MORE REFINED SKIN | 3.5* | 0.9 |
| CLEAR OF IMPURITIES | 2.8* | 0 |
| LIFTED SKIN | 3.2 | 4.0 |
| MORE EVEN SKIN TONE | 2.6* | 0 |
| MORE YOUTHFUL APPEARANCE | 1.9 | 0.8 |

TABLE 6
Skin Benefits Score After 2 Weeks (0-7, directional; not statistical)

| METRIC | INVENTIVE | BENCHMARK |
|---|---|---|
| SMOOTHER SKIN | 6.9* | 4.5 |
| MORE FIRM SKIN | 6.0 | 5.8 |
| HEALTHIER LOOKING SKIN | 6.4* | 3.3 |
| MORE RADIANT SKIN | 5.0* | 1.6 |
| IMPROVEMENT OVERALL TEXTURE | 5.8 | 5.0 |
| HYDRATED/MOISTURIZED | 5.6* | 1.6 |
| LOOK AND FEEL | | |
| COMFORTABLE/ | 3.1 | 4.7 |
| PLEASANT FEELING | | |
| MORE PLUMP/SOFT SKIN | 4.7 | 3.1 |
| LESS DULL SKIN APPEARANCE | 3.8* | 0.9 |
| MORE REFINED SKIN | 4.7* | 0.9 |
| CLEAR OF IMPURITIES | 2.8 | 2.7 |
| LIFTED SKIN | 3.7 | 1.9 |
| MORE EVEN SKIN TONE | 3.7 | 3.2 |
| MORE YOUTHFUL APPEARANCE | 2.6 | 2.2 |

TABLE 7
Skin Benefits Score After 4 Weeks (0-7, directional; not statistical)

| METRIC | INVENTIVE | BENCHMARK |
|---|---|---|
| SMOOTHER SKIN | 7.0 | 7.1 |
| MORE FIRM SKIN | 6.6 | 6.7 |
| HEALTHIER LOOKING SKIN | 5.8 | 6.3 |
| MORE RADIANT SKIN | 5.7* | 2.9 |
| IMPROVEMENT OVERALL TEXTURE | 5.7* | 3.1 |
| HYDRATED/MOISTURIZED | 4.6 | 2.7 |
| LOOK AND FEEL | | |
| COMFORTABLE/ | 4.1 | 4.3 |
| PLEASANT FEELING | | |
| MORE PLUMP/SOFT SKIN | 4.7* | 1.6 |
| LESS DULL SKIN APPEARANCE | 3.8 | 2.9 |
| MORE REFINED SKIN | 3.7 | 2.7 |
| CLEAR OF IMPURITIES | 3.7 | 4.2 |
| LIFTED SKIN | 3.0 | 2.6 |
| MORE EVEN SKIN TONE | 3.7 | 4.6 |
| MORE YOUTHFUL APPEARANCE | 2.6 | 4.7 |

### Example 5: Effect of pH on Composition After Mixing Parts

A study of the inventive composition was conducted in which the ratio of silicate and thickener was varied to effectively vary the pH of the composition. The inventors demonstrated that in embodiments with varied proportions of the two parts and pH in the range from 9 to 11 that above a pH of 11, the gel mask has a thin, liquidous texture that is drippy when applied to a substrate and that below pH 10.5, the gel forms visible gel chunks within the composition. The exemplified inventive composition as disclosed herein, having a ratio of cross-linked polyacrylic acid thickener to silicate of about 1:2.5 and a pH of about 10.8 produced a gel mask that was smooth and easily spreadable. Accordingly, within the disclosed and claimed ratio of polyacrylic acid thickener to silicate is a range from about 1:1 to 1:8 and pH in the range from 9 to 11, a gel mask forming composition having varying textural properties is provided.

### Example 6: In vivo Tests of Skin After Rinsing

In vivo testing on subjects was conducted to evaluate retention of hectorite on skin for conferring blurring effects, and to evaluate hydration effects after application to skin and subsequent rinsing of the Inventive 1 composition according to this disclosure.

Stratum corneum evaluation using confocal Raman (zscan) demonstrated post rinse retention of silica film within stratum corneum, posited to be encapsulating hectorite, with the inventive 1 composition.

Hydration was also evaluated using confocal Raman (zscan); comparison between Inventive 1 and benchmark demonstrated moisture performance is improved with the inventive composition as compared with benchmark.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“One or more,” as used herein, means at least one, and thus includes individual components as well as mixtures/combinations.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

Other than in the operating examples, or where otherwise indicated, all numbers expressing quantities of ingredients and/or reaction conditions may be modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” etcetera. All numbers, amounts, ranges, etcetera, are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While a number of exemplary aspects and embodiments have been discussed above, those of skill in the art will recognize certain modifications, permutations, additions, and sub-combinations thereof, and it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. It is therefore intended that the invention is not limited to the particular embodiments disclosed herein and that the following appended claims and claims hereafter introduced are interpreted to include all such modifications, permutations, additions, and sub-combinations as are within their true spirit and scope.

What I claim:

1. A cosmetic composition, comprising:
a. Combinable parts including
i. a lifting serum comprising at least one silicate and water; and
ii. a gelling solution comprising at least one cross-linked polyacrylic acid thickener; at least one clay; at least one glycosaminoglycan comprising a hyaluronic acid selected from sodium hyaluronate, hydrolyzed sodium hyaluronate, a hyaluronic acid derivative, or a combination thereof, the at least one glycosaminoglycan having a molecular weight in a range from about 1 million Dalton to about 1.4 million Dalton,
wherein, upon mixing of the combinable parts and application to a substrate, the composition forms a semi-solid gel film.

2. The cosmetic composition according to claim 1, wherein the cosmetic composition is free or essentially free of ingredients selected from the group consisting of polyol plasticizers, peptides, plant extracts, and combinations thereof.

3. The cosmetic composition according to claim 1, wherein the at least one silicate is selected from the group consisting of sodium silicate, magnesium silicate, calcium silicate, aluminum silicate, and combinations thereof.

4. The cosmetic composition according to claim 1, wherein the at least one cross-linked polyacrylic acid thickener comprises a carbomer.

5. The cosmetic composition according to claim 1, wherein the at least one glycosaminoglycan comprising a hyaluronic acid selected from sodium hyaluronate, hydrolyzed sodium hyaluronate, a hyaluronic acid derivative, or a combination thereof comprises sodium hyaluronate.

6. The cosmetic composition according to claim 1, wherein the at least one clay is selected from the group consisting of hectorite, montmorillonite, bentonite, attapulgite, sepiolite, talc, mica and combinations thereof.

7. The cosmetic composition according to claim 1, wherein the pH is between 9 and 11.

8. The cosmetic composition according to claim 1, wherein a ratio of cross-linked polyacrylic acid thickener to silicate is a range from about 1:1 to 1:8.

9. The cosmetic composition according to claim 1, comprising one or more one or more additives selected from salts, chelating agents, antimicrobial agents, neutralizing/pH-adjusting agents, surfactants, oils, waxes, butters, vitamins, fragrances, pearlescent agents, odor absorbers, coloring materials, essential oils, and emulsifiers, fruit extracts, skin actives, and combinations thereof.

10. The cosmetic composition according to claim 1, wherein the at least one silicate is present in a range from about 1% to about 15%, the at least one thickener is present in a range from about 1% to about 2%, the at least one glycosaminoglycan comprising a hyaluronic acid is present is present in a range from about 0.01% to about 0.1%, the at least one clay is present in a range from about 0.02% to about 0.1%, and water is present in a range from about 89% to about 98%, all amounts by weight, based on the weight of the cosmetic composition.

11. The cosmetic composition according to claim 1, wherein the pH is about 10.8

12. A cosmetic composition, comprising:
a. Combinable parts including
i. a lifting serum comprising at least one silicate and water; and
ii. a gelling solution comprising at least one cross-linked polyacrylic acid thickener; at least one clay; at least one glycosaminoglycan comprising a hyaluronic acid selected from sodium hyaluronate, hydrolyzed sodium hyaluronate, a hyaluronic acid derivative, or a combination thereof, the at least one glycosaminoglycan having a molecular weight in a range from about 1 million Dalton to about 1.4 million Dalton,
wherein, upon mixing of the combinable parts and application to a substrate, the composition forms a semi-solid gel film, and
wherein the at least one silicate comprises a sodium silicate present at about 3.3%, water is present at about 94% by weight, the at least one thickener comprises a carbomer, present at about 1.3%, the at least one glycosaminoglycan comprises sodium hyaluronate present at about 0.07%, the at least one clay comprises hectorite present at about 0.05% by weight, and the cosmetic composition includes butylene glycol is present at about 0.3%, all amounts by weight based on the weight of the composition.

13. The cosmetic composition according to claim 12, wherein the cosmetic composition is free or essentially free of ingredients selected from the group consisting of polyol plasticizers, peptides, plant extracts, and combinations thereof.

14. An article of manufacture comprising a composition according to claim 1, wherein the article of manufacture includes a first container retaining the lifting serum part (i) of the composition and a second container retaining the gelling solution (ii) of the composition.

15. The article of manufacture according to claim 14, the article of manufacture characterized by one of: (a) the first and second containers are provided for dispensing multiple doses of the composition, wherein one of the first and second containers is sized to receive the contained contents of both the first and second containers, such that the contained parts may be blended and used over time until the composition is exhausted; (b) the first and second containers are formed as discrete chambers of a single package have a frangible seal between them, such that the seal may be broken to permit mixing of the contents of the first and second containers, to thereby provide a single or plurality of doses; (c) the first and second containers are formed as discrete pouches of a single package; or (d) the first and second containers are formed as discrete pouches, jars, packets, compacts, bottles, tubes, or ampoules.

16. The article of manufacture according to claim 14, comprising an applicator brush.

17. An article of manufacture comprising a composition according to claim 12, wherein the article of manufacture includes a first container retaining the lifting serum part (i) of the composition and a second container retaining the gelling solution (ii) of the composition.

18. The article of manufacture according to claim 17, the article of manufacture characterized by one of: the first and second containers are provided for dispensing multiple doses of the composition, wherein one of the first and second containers is sized to receive the contained contents of both the first and second containers, such that the contained parts may be blended and used over time until the composition is exhausted; the first and second containers are formed as discrete chambers of a single package have a frangible seal between them, such that the seal may be broken to permit mixing of the contents of the first and second containers, to thereby provide a single or plurality of doses; the first and second containers are formed as discrete pouches of a single package; or the first and second containers are formed as discrete pouches, jars, packets, compacts, bottles, tubes, or ampoules.

19. The article of manufacture according to claim 17, comprising an applicator brush.

20. A method of forming a gel mask on a substrate, comprising: providing a composition according to claim 1, mixing the component parts of the composition for 10-30 seconds; applying the mixture to the substrate for a period of time from about 10 to about 25 minutes; rinsing the substrate with warm water.
